(19) United States
(12) Patent Application Publication
(10) Pub. No.: US 2019/0218307 A1
(43) Pub. Date: Jul. 18, 2019

(54) TIRC7 BASED DIAGNOSTIC AND THERAPY OF CANCER

(71) Applicant: Nekonal S.a.r.l., Luxembourg (LU)

(72) Inventor: Nalan UTKU, Munich (DE)

(21) Appl. No.: 16/324,385

(22) PCT Filed: Aug. 9, 2017
(86) PCT No.: PCT/EP2017/070222

§ 371 Date: Feb. 8, 2019

(30) Foreign Application Priority Data
Aug. 9, 2016 (EP) 16183425.4

(51) Int. Cl. C07K 16/40; A61P 35/02; C07K 16/28; G01N 33/574

(52) U.S. Cl. CPC C07K 16/40; G01N 33/57426; C07K 16/28; A61P 35/02

(57) ABSTRACT

The present invention pertains to a combined diagnostic and therapeutic approach for leukemia/lymphoma patients comprising the analysis of the biomarker T cell immune response cDNA 7 (TIRC7) and, depending on its expression in leukemia/lymphoma cells in a patient, the use of TIRC7 as a target for therapy. In particular, the invention provides methods for stratifying leukemia/lymphoma patients into two groups one of which will benefit from a TIRC7 modulatory treatment, and one of which are non-responders to such a therapy. Furthermore, the invention provides compounds for the treatment of patients that are identified as responders according to the invention. Thus, the present disclosure offers a true theranostic approach for leukemia/lymphoma patients based on TIRC7.

### FIELD OF THE INVENTION

The present invention pertains to a combined diagnostic and therapeutic approach for leukemia/lymphoma patients comprising the analysis of the biomarker T cell immune response cDNA 7 (TIRC7) and, depending on its expression in leukemia/lymphoma cells in a patient, the use of TIRC7 as a target for therapy. In particular, the invention provides methods for stratifying leukemia/lymphoma patients into two groups, one of which will benefit from a TIRC7 modulatory treatment, and one of which are non-responders to such a therapy. Furthermore, the invention provides compounds for the treatment of patients that are identified as responders according to the invention. Thus, the present disclosure offers a true theranostic approach for leukemia/lymphoma patients based on TIRC7.

### DESCRIPTION

Estimates of the worldwide incidence, mortality and prevalence of cancer in the year 2002, show 10.9 million new cases, 7.6 million deaths, and 24.6 million persons alive with cancer [Parkin DM 2005]. Leukaemia accounts for some 300.000 new cases each year (2.8% of all new cancer cases) [Parkin DM 2005] and causes about 3% of the almost seven million deaths due to progression. In 2000, approximately 256,000 children and adults around the world developed a form of leukemia, and 209,000 died from the progressive disease.

Leukemias comprise a heterogeneous group of clonal hematologic malignancies which continue to cause significant mortality and morbidity despite decades of research and drug development. Leukemias are classified as acute or chronic, depending upon their clinical development and expected progression, and as lymphoid or myeloid, depending upon the lineage from which the cancer arises. The acute leukemias are characterized by a rapidly progressive, fatal course if untreated, but these diseases often respond to aggressive cytotoxic chemotherapy, especially in children and younger adults. Acute lymphocytic leukemia (ALL) affects both children and adults, while acute myelogenous leukemia (AML) strikes predominantly adults. Although great strides in clinical response and long-term survival were initially achieved with the advent of chemotherapy in leukemia patients, particularly children, a plateau in clinical benefit has been reached with the use of standard forms of cytotoxic chemotherapy alone [Lin, T S 2000]. AML is a relatively rare cancer. There are approximately 3.600 new cases each year in Germany. The incidence of AML increases with age; the median age at diagnosis is 63 years. AML accounts for about 80% of all acute leukemias in adults, but is with 15-20% of acute leukemias rare in children. AML is slightly more common in men, with a male-to-female ratio of 1.4:1 [http://seers.cancer.gov/seers.cancer.gov]. The rate of therapy-related AML (AML caused by previous chemotherapy) is rising; therapy-related disease currently accounts for about 10-20% of all cases of AML [Greenley, Jemal, Linet)]. AML is characterised by the proliferation of clonal precursor myeloid cells with arrested differentiation and subsequent accumulation of myeloid blasts in the bone marrow. Approximately 60%-80% of younger adults with AML achieve complete remission (CR) with conventional chemotherapy such as cytarabine and an anthracycline. However, a significant proportion of the responsive patients suffer relapses and die of treatment-refractory disease. The treatment of relapsed AML patients is considerably less successful, especially in the elderly, because the toxicity of standard induction chemotherapy is poorly tolerated in the older age group.

Thus, novel drugs and treatment strategies are major objectives of research; conjugates of antibodies with powerful cytotoxic agents have been explored. Gemtuzumab-ozogamicin (GO) is the first immunoconjugate approved by the United States Food and Drug Administration (FDA) for treating refractory AML. A novel immunoconjugate, AVE9633, has been evaluated in Phase I clinical trials on refractory AML patients which provided initial evidence that AVE9633 has anti-leukaemia activity. Due to limited availability of patients in AML and relatively small market, there is only restricted effort of pharmaceutical industry to develop novel therapies.

ALL is a rare cancer with approximately 500 new cases in adults and 500 in children each year in Germany. The median age at diagnosis is 13 years. ALL accounts for less than 15% (more than 85% of leukemia cases in adults are AML). However, in children it is the most common type of childhood cancer. ALL is like AML slightly more common in men, with a male-to-female ratio of 1.4:1 [http://http://seers.cancer.gov/seers.cancer.gov]. ALL is a heterogeneous disorder deriving from transformation of hematopoietic stem cells and, possibly, from lymphoid-committed progenitor cells with the greatest prevalence in children, but it also affects adults, and has an increasing incidence with age. Chromosomal abnormalities in ALL have been frequently described, the most common is the Philadelphia chromosome (Ph). The resulting fusion gene, BCR-ABL1, encodes for a chimerical oncoprotein (BCR-ABL) with constitutive tyrosine kinase activity, which leads to uncontrolled cell proliferation, reduced apoptosis, and impaired cell adhesion. Treating Philadelphia chromosome-positive (Ph1) ALL patients with conventional chemotherapy has not substantially improved their long-term outcomes, but it was the only treatment option until the availibility of imatinib [Piccaliga 2007]. Recently, however, BCR-ABL-targeted strategies have been successfully adopted. Imatinib is an oral competitive inhibitor of ABL with demonstrated phase 2 efficacy in patients with treatment-naive and pretreated ALL. Despite its efficacy, imatinib may induce specific resistance in a large proportion of patients, mainly because of the occurrence of ABL1 mutations. Therefore, novel inhibitors like dasatinib have been developed, but are still being evaluated in clinical trials. Thus, in many cancer indications “old-fashioned” chemotherapeutics still maintain their dominating position as cancer agents for first-line and second-line therapy in many cases.

A potential candidate for the development of novel therapeutic strategies is the cell surface protein, TIRC7 (T cell immune response cDNA7) induced after immune activation in subset of human T-, B-cells as well as monocytes. During immune activation, TIRC7 is colocalized with the T cell receptor and CTLA4 within the immune synapse of human T cells. At protein and mRNA level, its expression is induced in lymphocytes in synovial tissues obtained from patients with rheumatoid arthritis or during rejection of solid organ transplants and bone marrow transplantation as well as in brain tissues obtained from patients with multiple sclerosis.

In view of the above drawback in the diagnosis and treatment of lymphoid or leukemic malignancies, it was an object of the present invention to provide novel combined diagnostic and therapeutic approaches for patients. The invention seeks to provide novel options for late stage non-Hodgkin lymphoma (NHL) patients, in particular such patients that already received anti-CD20 therapy and have become refractory, and/or have become resistant to an anti-CD20 therapy.

Thus, the above problem is first solved by a method for stratifying a leukemia/lymphoma patient into one of patient groups (i) or (ii), wherein patient group (i) is a leukemia/lymphoma patient group that will benefit from a T cell immune response cDNA 7 (TIRC7)-modulatory treatment, and patient group (ii) is a leukemia/lymphoma patient group that will not benefit from a TIRC7 modulatory treatment, the method comprising the method steps of
(a) Providing a sample comprising a leukemia/lymphoma tumor cell from said leukemia/lymphoma patient,
(b) Determining the expression of TIRC7 in or on the leukemia/lymphoma tumor cell,
(c) Depending on the resultant of step (b), stratifying the patient into group (i) in the event the leukemia/lymphoma tumor cell expresses TIRC7 compared to a control cell, or stratifying the patient into group (ii) in the event the leukemia/lymphoma tumor cell does not express TIRC7 compared to a control cell.

In an alternative aspect the invention provides a method for diagnosing a leukemia/lymphoma patient to have a leukemia/lymphoma with which the patient will benefit from a TIRC7 modulatory treatment, the method comprising the method steps of
(a) Providing a sample comprising a leukemia/lymphoma tumor cell from said leukemia/lymphoma patient,
(b) Determining the expression of TIRC7 in or on the leukemia/lymphoma tumor cell,
(c) Depending on the resultant of step (b), diagnosing the patient to have a lymphoma which is treatable by administering a TIRC7 modulator to the patient in the event the leukemia/lymphoma tumor cell expresses TIRC7 compared to a control cell.

The present invention is based on the finding that leukemia/lymphoma patients surprisingly show different cancer cell expression of TIRC7, a major key regulator of proliferative pathways in immune cells. According to the herein disclosed invention, TIRC7 can be used to stratify leukemia/lymphoma cancers into a responder and a non-responder group with respect to a TIRC7 modulatory treatment of the disease. Therefore, the present invention for the first time provides a true theranostic approach (a combined diagnostic and therapeutic approach) for leukemia/lymphoma patients.

The methods of the invention may additionally in step (b) comprise: determining the expression of TIRC7 together with at least one additional immune cell factor selected from the group consisting of FoxP3 and CD20.

In context of the present invention it may be preferred that TIRC7 modulatory treatment is a TIRC7 treatment that involves the activation of an TIRC7 dependent apoptotic signalling cascade. TIRC7 may induce apoptosis via signalling initiated by the binding of the TIRC7 ligand HLA DR alpha 2. Therefore in preferred embodiments of the herein described invention the TIRC7 modulatory treatment comprises the activation of the TIRC7-HLA-DR alpha 2 axis or signalling.

Correspondingly, the invention with respect to the term “TIRC7 modulator” preferably refers to a compound, which is selected from any compound that modulates TIRC7 expression, stability and/or function, and even more preferably is a compound, which modulates the signalling of the TIRC7 HLA-DR alpha 2 axis. Such a compound preferably is an inhibitor of lymphoma/leukemia cell proliferation or growth. A TIRC7 modulator may in some preferred embodiments of the invention be selected from compounds that mimic HLA-DR alpha 2 mediated TIRC7 signalling. Such TIRC7 modulators may be selected from a nucleic acid, a protein, or a small compound. Preferred inhibitors of TIRC7 of the invention include anti-TIRC7 antibodies, such as for example any of the antibodies disclosed in WO 99/11782, WO 03/054019 and WO 03/054018 (all incorporated herein in their entirety). A preferred antibody is Metiliximab (disclosed in WO 03/054019) as well as any chimerized, humanized or otherwise derivatized variants or fragments thereof, wherein the variant still comprises the CDR1 to CDR3 regions of the parent molecule. Most preferably, the invention pertains to Metiliximab, or any derivatives and chimeras thereof, as a TICR7 modulator. Alternatively, the invention as TIRC7 modulator provides HLA-DR alpha 2 encoding nucleic acids or recombinant proteins, or functional variants or fragments thereof.

In some embodiments, the herein disclosed methods are ex vivo or in vitro methods, preferably wherein the methods are conducted completely ex vivo or in vitro.

The present invention provides a combined diagnostic and therapeutic approach for leukemia/lymphoma patients. In context of the present disclosure, the lymphoma is preferably selected from Hodgkin lymphoma or non-Hodgkin lymphoma (NHL), however, wherein NHL is most preferred. The leukemia is preferably selected from CML, AML or ALL.

In some embodiments, it is preferred that the leukemia/lymphoma is a leukemia/lymphoma disease in a late stage and/or a refractory leukemia/lymphoma. A refractory leukemia/lymphoma may be a leukemia/lymphoma disease that was already treated with state of the art chemotherapeutic method, and which has developed a resistance to chemotherapeutic agents selected from agents excluding TIRC7 modulators as defined and described in the present disclosure. In some preferred embodiments of the present invention the leukemia/lymphoma is a refractory disease with a resistance to a treatment targeting the protein CD20, such as an anti-CD20 antibody treatment, most preferably a leukemia/lymphoma resistant to a treatment with rituximab or check point inhibitors e.g. PD1. PDL1 or CTLA4

In context of the herein disclosed invention the term “non-Hodgkin lymphoma” shall include the following disorders, which may each be a lymphoma preferred for the herein disclosed invention: pre-cursor or mature B cell neoplasms, T cell and natural killer cell neoplasms, for example diffuse large B-cell lymphoma (DLBCL), B-cell prolymphocytic leukemia, lymphoplasmacytic lymphoma (such as Waldenstrom macroglobulinemia), splenic marginal zone lymphoma, plasma cell neoplasms, extranodal marginal zone B cell lymphoma (also called MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, and Burkitt lymphoma/leukemia, whereas T cell and natural killer (NK) cell neoplasms are exemplified by T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, angioimmunoblastic T cell lymphoma, peripheral T cell lymphoma, unspecified, CMML, plasmacytoma (Morbus Kahler or Multiples Myeloma) and anaplastic large cell lymphoma.

For the purpose of the present invention the term leukemia generally refers to any of the various types and subtypes of leukemia, i.e. lymphoblastic leukemia (ALL), acute myeloid leukemia (AML), chronic lymphocytic leukemia (CLL) and chronic myeloid leukemia (CML), and any subtype of these, as well as any of the other, less common types of leukemia.

The diagnostic approach of the present invention requires in step (b) a determining of the expression of TIRC7 in or on the leukemia/lymphoma tumor cell. In some preferred embodiment of the invention the TIRC7 expression may be determined on the TIRC7 protein level, for example by using anti-TIRC7 antibodies or mass spectroscopic methods, or at the mRNA level, for example using a PCR-based detection method or nucleic acid based hybridization technique. Any method known to the skilled person to determine the expression of a protein on a cell may be used in context of the present invention.

In preferred embodiments of the present invention, said control cell used in the methods is a cell not expressing TIRC7 protein (therefore is a negative control). Alternatively to a negative control, the method of the invention may comprise a comparison of the determined level of TIRC7 expression on the tumor cell with a reference value of a TIRC7 expression value that corresponds to the level of TIRC7 expression in a negative control.

A sample according to the present disclosure which comprises a leukemia/lymphoma tumor cell is preferably a tissue sample or liquid sample, wherein the tissue sample is obtained from a lymph node, and wherein the liquid sample may be a blood sample.

In some embodiments of the present disclosure the leukemia/lymphoma patient is a relapsed or refractory leukemia/lymphoma patient in which a previous therapy with a compound selected from the group consisting of an anti-CD20 antibody such as Rituximab; or fludarabine and chlorodeoxiadenosine, has failed, or wherein the leukemia/lymphoma is refractory and has become resistant to any of the aforementioned treatments.

Another aspect of the invention then pertains to a modulator of T cell immune response cDNA 7 (TIRC7) for use in the treatment of leukemia/lymphoma in a patient. In this aspect, the above mentioned definitions equally apply. In particular, in some embodiments it is preferred that the leukemia/lymphoma is a leukemia/lymphoma disease in a late stage and/or a refractory leukemia/lymphoma. A refractory leukemia/lymphoma may be a leukemia/lymphoma disease that was previously treated with state of the art chemotherapeutic method either successfully or not, and which subsequently developed a resistance to chemotherapeutic agents selected from chemotherapeutic agents excluding TIRC7 modulators as defined and described in the present disclosure. In some preferred embodiments of the present invention, the leukemia/lymphoma is a refractory disease with a resistance to treatment targeting the protein CD20, such as anti-CD20 antibody treatment, most preferably a leukemia/lymphoma resistant to a treatment with rituximab.

In context of the present disclosure, the treatment preferably comprises a preceding stratification of the patient suffering from leukemia/lymphoma. Such stratification is a stratification of the patient into one of patient groups (i) or (ii), wherein patient group (i) is a leukemia/lymphoma patient group that will benefit from a T cell immune response cDNA 7 (TIRC7)-modulatory treatment, and patient group (ii) is a leukemia/lymphoma patient group that will not benefit from a TIRC7 modulatory treatment. The stratification is preferably a method as disclosed herein above.

Also provided are pharmaceutical compositions for use in the herein disclosed medical applications, comprising a TIRC7 modulator as described before, together with a pharmaceutical acceptable carrier and or excipient. The term “pharmaceutical composition” refers to a preparation which is in such form as to permit the biological activity of an active ingredient contained therein to be effective, and which contains no additional components which are unacceptably toxic to a subject to which the composition would be administered. A pharmaceutical composition of the present invention can be administered by a variety of methods known in the art. As will be appreciated by the skilled artisan, the route and/or mode of administration will vary depending upon the desired results. To administer an antibody according to the invention by certain routes of administration, it may be necessary to coat the antibody with, or co-administer the antibody with, a material to prevent its inactivation. For example, the antibody may be administered to a subject in an appropriate carrier, for example, liposomes, or a diluent. Pharmaceutically acceptable diluents include saline and aqueous buffer solutions. A “pharmaceutically acceptable carrier” refers to an ingredient in a pharmaceutical formulation, other than an active ingredient, which is nontoxic to a subject. Pharmaceutically acceptable carriers includes any and all solvents, dispersion media, coatings, antibacterial and antifungal agents, isotonic and absorption delaying agents, and the like that are physiologically compatible. In one preferred embodiment, the carrier is suitable for intravenous, intramuscular, subcutaneous, parenteral, spinal or epidermal administration (e.g. by injection or infusion).

The pharmaceutical compositions according to the invention may also contain adjuvants such as preservatives, wetting agents, emulsifying agents and dispersing agents. Prevention of presence of microorganisms may be ensured both by sterilization procedures, supra, and by the inclusion of various antibacterial and antifungal agents, for example, paraben, chlorobutanol, phenol, sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Regardless of the route of administration selected, the compounds of the present invention, which may be used in a suitable hydrated form, and/or the pharmaceutical compositions of the present invention, are formulated into pharmaceutically acceptable dosage forms by conventional methods known to those of skill in the art. Actual dosage levels of the active ingredients in the pharmaceutical compositions of the present invention may be varied so as to obtain an amount of the active ingredient, which is effective to achieve the desired therapeutic response for a particular patient, composition, and mode of administration, without being toxic to the patient. The selected dosage level will depend upon a variety of pharmacokinetic factors including the activity of the particular compositions of the present invention employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

The composition must be sterile and fluid to the extent that the composition is deliverable by syringe. In addition to water, in one embodiment the carrier is an isotonic buffered saline solution. Proper fluidity can be maintained, for example, by use of coating such as lecithin, by maintenance of required particle size in the case of dispersion and by use of surfactants. In many cases, it is preferable to include isotonic agents, for example, sugars, polyalcohols such as mannitol or sorbitol, and sodium chloride in the composition. An “immunoconjugate” is an antibody conjugated to one or more heterologous molecule(s), including but not limited to a cytotoxic agent.

The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently. A typical dose can be, for example, in the range of 0.001 to 1000 μg (or of nucleic acid for expression or for inhibition of expression in this range); however, doses below or above this exemplary range are envisioned, especially considering the aforementioned factors. Generally, the regimen as a regular administration of the pharmaceutical composition should be in the range of 1 μg to 10 mg units per day. If the regimen is a continuous infusion, it should also be in the range of 1 μg to 10 mg units per kilogram of body weight per minute, respectively. Progress can be monitored by periodic assessment. Dosages will vary but a preferred dosage for intravenous administration of DNA is from approximately 10<6> to 10<12> copies of the DNA molecule. The compositions of the invention may be administered locally or systemically. Administration will generally be parenterally, e.g., intravenously; DNA may also be administered directly to the target site, e.g., by biolistic delivery to an internal or external target site or by catheter to a site in an artery. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, antioxidants, chelating agents, and inert gases and the like. Furthermore, the pharmaceutical composition of the invention may comprise further agents such as interleukins or interferons depending on the intended use of the pharmaceutical composition.

In another aspect, the invention pertains to a kit, comprising means for use in the combined diagnostic and therapeutic approach of the present invention. The kit preferably comprises (a) means for the determination of TIRC7 expression and (b) a TIRC7 modulator as a therapeutic. Optionally the kit may further comprise instructions for use and/or other agents that are helpful in the diagnosis or therapy of leukemia/lymphoma.

Furthermore provided are methods for the treatment of a patient suffering from a leukemia/lymphoma disorder, the method comprising a step of administering a therapeutically effective amount of an TIRC7 modulator as described herein before. Preferably, the TIRC7 modulator is a TIRC7 ligand, or derivative thereof, or an anti-TIRC7 antibody, or derivative or antigen binding fragment thereof, as described above.

The present invention will now be further described in the following examples with reference to the accompanying figures and sequences, nevertheless, without being limited thereto. For the purposes of the present invention, all references as cited herein are incorporated by reference in their entireties. In the Figures:

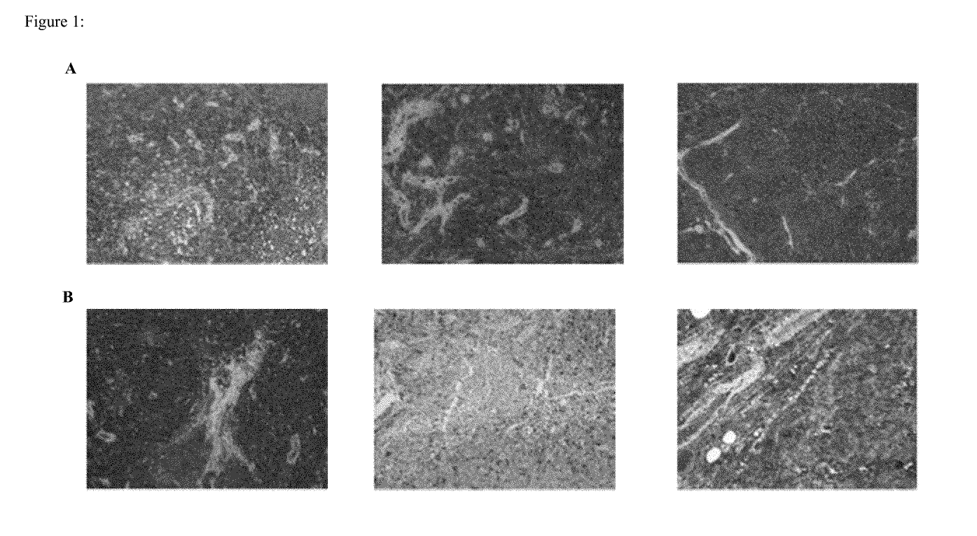

FIG. 1: TIRC7 expression is induced in human lymphoma cells: preparations from left to right: A: DGB diffuse large cell Lymphoma. B-NHL: B cell non-hogkin cell Lymphoma; MZL: Mantel cell Lymphoma; FL follicular Lymphoma; B: BCLL chron. lymphat. Leukemia; ALCL: anaplastic large cell Lymphoma; LB lymphoblastic Lymphoma.

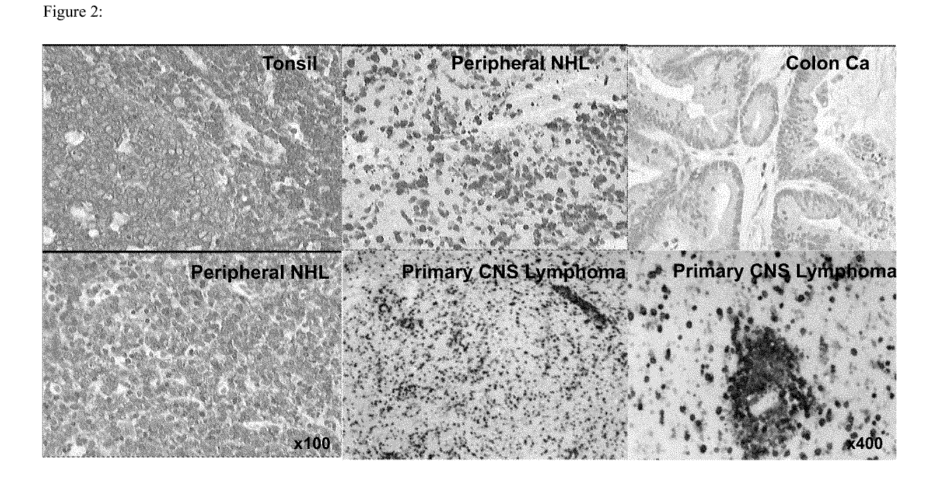

FIG. 2: TIRC7 expression is induced on human peripheral lymphoma and primary central lymphomas.

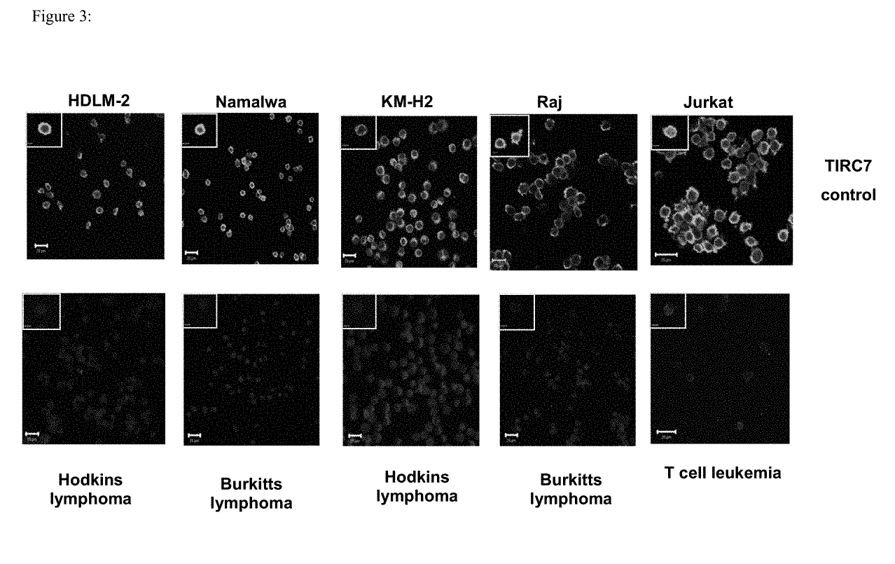

FIG. 3: TIRC7 expression in various human lymphoma cell lines.

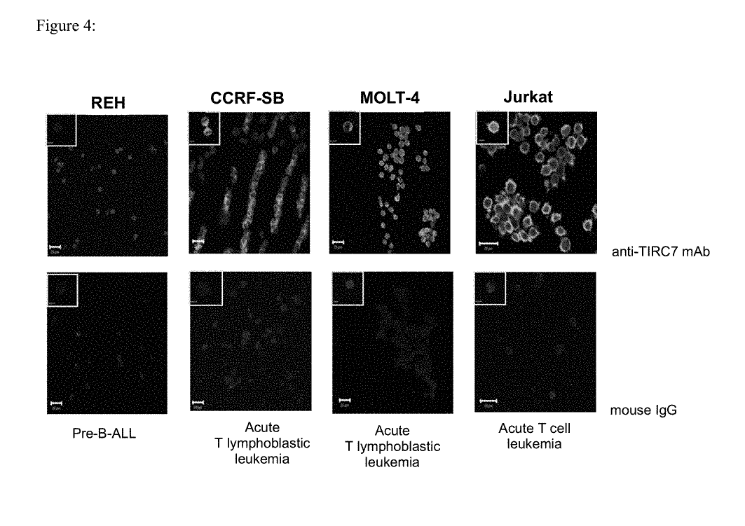

FIG. 4: TIRC7 expression in human acute leukemia cell lines.

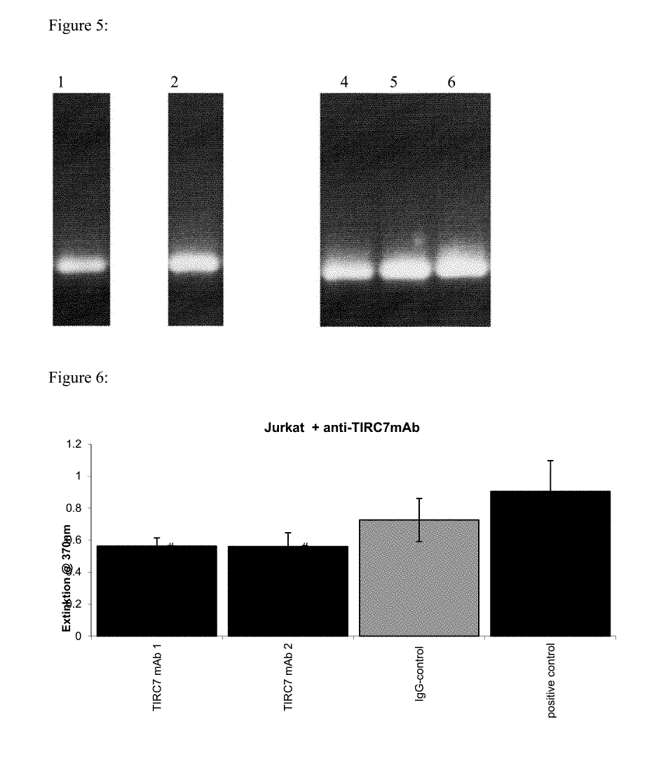

FIG. 5: TIRC7-mRNA is induced in human acute leukemia cell lines.

FIG. 6: Anti-TIRC7 mAbs inhibit proliferation of human leukemia cell line

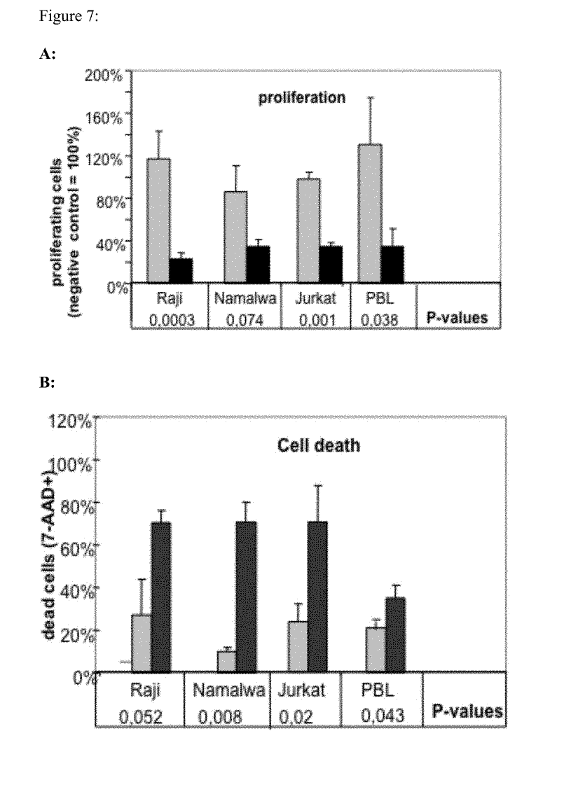

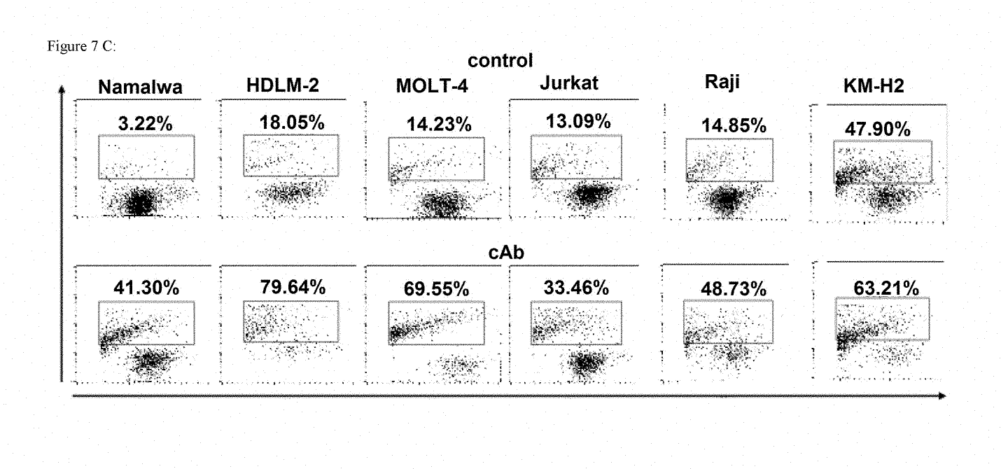

FIG. 7: Anti-TIRC7 chimeric mAb (cAb) induces apoptosis and inhibits proliferation of various tumor cell lines (black bars); A: cell proliferation; B: cell death; C: FACS results.

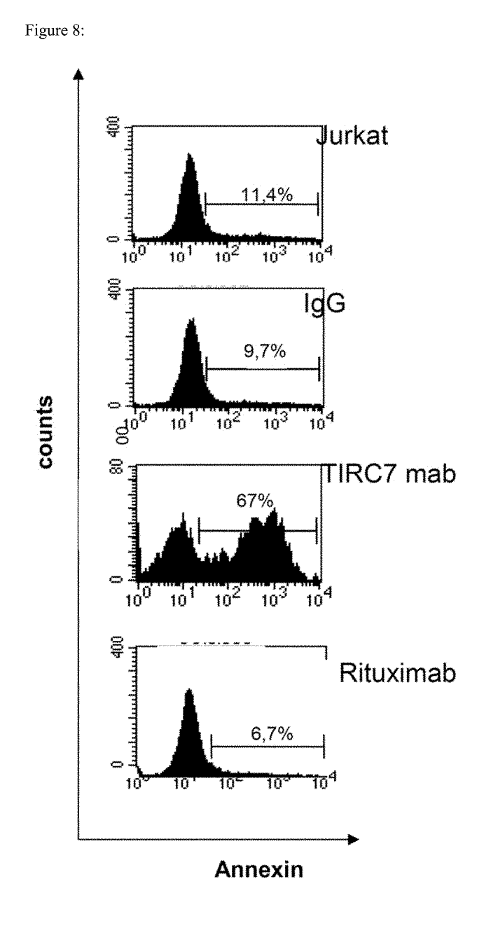

FIG. 8: Anti-TIRC7 chimeric mAb leads also to high apoptosis in Jurkat cells whereas Rituximab has no effect.

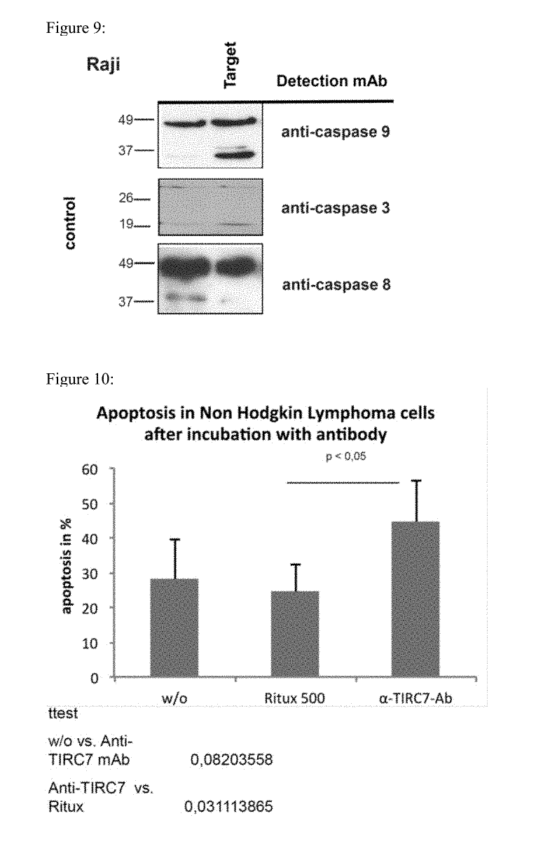

FIG. 9: Anti-TIRC7 mAb leads to apoptosis via the induction of caspase 9 in B cell lymphoma (Raji) cells.

FIG. 10: Peripheral lymphoma cells isolated from patients showed a significant increase of apoptosis after incubation (ex-vivo) with anti-TIRC7 mAb in comparison to controls.

### EXAMPLES

In FIG. 1 Anti-Tirc7 antibody (Metiliximab) was used to stain tumors derived from various patients with lymphoma according to standard immune histo staining procedures of formalin embedded tissues (Bulwin et al, Plos One 2007). In FIG. 2 Anti-Tirc7 antibodies were used to stain tumors from patients with brain lymphoma (Bulwin et al, Plos One 2007). As can be seen from the results, TIRC7 is expressed in these lymphoma cells.

In order to test whether TIRC7 is also expressed in further lymphomas, anti-Tirc7 antibody was used to stain cell lines obtained from various human lymphoma samples (Bulwin et al, Plos One 2007). The results are provided in FIG. 3. Also, TIRC7 is expressed in various leukemia cell lines (FIG. 4) and is induced in human acute leukemia cell lines (FIG. 5).

Based on the surprising results that TIRC7 is expressed in some leukemia patients, and not in others, it was evaluated whether the treatment with anti-TIRC7 mAbs could inhibit proliferation of human leukemia cell lines. To examine the inhibitory effect of the anti-TIRC7 mAb on human leukemia, primary cell lines were incubated in the presence and absence of Anti-Tirc7 antibody or control antibody and subjected to proliferation assays. The results in FIG. 6 show the inhibition of proliferation of leukemia cell lines indicating a therapeutic use of TIRC7 modulators in leukemia and lymphoma.

To further examine the inhibitory effect of the anti-TIRC7 mAb on human leukemia and lymphoma cell lines were incubated in the presence and absence of anti-Tirc7 antibody or control antibody and subjected simultaneously to proliferation and apoptosis assays using flow cytometry method (FIG. 7).

To examine the apoptotic effect of the anti-TIRC7 mAb on human T cell lymphoma cell line (Jurkat), cells were incubated in the presence and absence of chimeric anti-Tirc7 antibody or control antibody or chimeric antibody Rituxan and subjected to apoptosis assays using flow cytometry method (FIG. 8).

To examine the apoptotic effect of the anti-TIRC7 mAb on human B cell lymphoma cell line (Raji), cells were incubated in the presence and absence of chimeric anti-Tirc7 antibody or control antibody and subjected to caspase assay using caspase specific antibodies (Bulwin et al, PlosOne 2007). The results are provided in FIG. 9.

The inhibitory effect of the anti-TIRC7 mAb on primary human lymphoma cells freshly isolated from patients and incubated in the presence and absence of anti-Tirc7 antibody or control antibody was confirmed in ex vivo proliferation assays (FIG. 10).

1. An ex vivo method for stratifying a leukemia/lymphoma patient into one of patient groups (i) or (ii), wherein patient group (i) is a leukemia/lymphoma patient group that will benefit from a T cell immune response cDNA 7 (TIRC7)-modulatory treatment, and patient group (ii) is a leukemia/lymphoma patient group that will not benefit from a TIRC7 modulatory treatment, the method comprising the ex vivo method steps of
(a) Providing a sample comprising a leukemia/lymphoma tumor cell from said leukemia/lymphoma patient,
(b) Determining the expression of TIRC7 in or on the leukemia/lymphoma tumor cell,
(c) Depending on the resultant of step (b), stratifying the patient into group (i) in the event the leukemia/lymphoma tumor cell expresses TIRC7 compared to a control cell, or stratifying the patient into group (ii) in the event the leukemia/lymphoma tumor cell does not express TIRC7 compared to a control cell.

2. An ex vivo method for diagnosing a leukemia/lymphoma patient to have a leukemia/lymphoma with which the patient will benefit from a TIRC7 modulatory treatment, the method comprising the ex vivo method steps of
(a) Providing a sample comprising a leukemia/lymphoma tumor cell from said leukemia/lymphoma patient,
(b) Determining the expression of TIRC7 in or on the leukemia/lymphoma tumor cell,
(c) Depending on the resultant of step (b), diagnosing the patient to have a leukemia/lymphoma which is treatable by administering a TIRC7 modulator to the patient in the event the leukemia/lymphoma tumor cell expresses TIRC7 compared to a control cell.

3. The method according to claim 1 or 2, wherein step (b) comprises determining the expression of TIRC7 and at least one additional immune cell factor selected from the group consisting of FoxP3 and CD20.

4. The method according to any of claims 1 to 3, wherein TIRC7 modulatory treatment is an TIRC7 inhibitory treatment and wherein said TIRC7 modulator is a TIRC7 inhibitor.

5. The method according to any of claims 1 to 4, which is performed completely ex vivo, preferably in vitro.

6. The method according to any of claims 1 to 5, wherein the TIRC7 modulatory treatment is a treatment comprising the administration of a TIRC7 modulator, such as an antibody binding to, and modulating, TIRC7, preferably wherein the antibody is neliximab or metiliximab, or antibody derivatives of these molecules.

7. The method according to any of claims 1 to 6, wherein the lymphoma is selected from Hodgkin lymphoma or non-Hodgkin lymphoma.

8. The method according to claim 7, wherein the non-Hodgkin lymphomas include pre-cursor or mature B cell neoplasms, T cell and natural killer cell neoplasms, for example diffuse large B-cell lymphoma (DLBCL), B-cell prolymphocytic leukemia, lymphoplasmacytic lymphoma (such as Waldenstrom macroglobulinemia), splenic marginal zone lymphoma, plasma cell neoplasms, extranodal marginal zone B cell lymphoma (also called MALT lymphoma), nodal marginal zone B cell lymphoma (NMZL), follicular lymphoma, mantle cell lymphoma, diffuse large B cell lymphoma, mediastinal (thymic) large B cell lymphoma, intravascular large B cell lymphoma, primary effusion lymphoma, and Burkitt lymphoma/leukemia, whereas T cell and natural killer (NK) cell neoplasms are exemplified by T cell prolymphocytic leukemia, T cell large granular lymphocytic leukemia, aggressive NK cell leukemia, adult T cell leukemia/lymphoma, extranodal NK/T cell lymphoma, nasal type, enteropathy-type T cell lymphoma, hepatosplenic T cell lymphoma, blastic NK cell lymphoma, mycosis fungoides/Sezary syndrome, primary cutaneous CD30-positive T cell lymphoproliferative disorders, angioimmunoblastic T cell lymphoma, peripheral T cell lymphoma, unspecified, CMML, plasmacytoma (Morbus . . . ) and anaplastic large cell lymphoma.

9. The method according to any of claims 1 to 8, wherein in step (b) the expression of TIRC7 in or on the leukemia/lymphoma tumor cell is determined on the protein level, for example by using an anti-TIRC7 antibody, or at the mRNA level, for example by a PCR-based detection method or hybridization technique.

10. The method according to any of claims 1 to 9, wherein said control cell is a cell not expressing TIRC7 protein (negative control).

11. The method according to any of claims 1 to 10, wherein the sample comprising a leukemia/lymphoma tumor cell is a tissue sample or liquid sample, wherein the tissue sample is obtained from a lymph node, and wherein the liquid sample may be a blood sample.

12. The method according to any of claims 1 to 11, wherein the leukemia/lymphoma patient is a relapsed or refractory leukemia/lymphoma patient in which a previous therapy with a compound selected from the group consisting of an anti-CD20 antibody such as Rituximab, fludarabine and chlorodeoxiadenosine, has failed.

13. A modulator of T cell immune response cDNA 7 (TIRC7) for use in the treatment of leukemia/lymphoma in a patient.

14. The modulator of TIRC7 for use according to claim 13, wherein the modulator of TIRC7 is an antibody binding to, and inhibiting, the extracellular domain of TIRC7, preferably wherein the antibody or antibody derivatives of these molecules, such as chimerized, humanized, or otherwise optimized antibody molecules.

15. The modulator of TIRC7 for use according to claim 13 or 14, wherein the leukemia/lymphoma is a TIRC7 expressing leukemia/lymphoma.

16. The modulator of TIRC7 for use according to any of claims 13 to 15, wherein the treatment comprises a preceding stratification of the patient suffering from leukemia/lymphoma.

17. The modulator of TIRC7 for use according to claim 16, wherein the stratification is a stratification of the patient into one of patient groups (i) or (ii), wherein patient group (i) is a leukemia/lymphoma patient group that will benefit from a T cell immune response cDNA 7 (TIRC7)-modulatory treatment, and patient group (ii) is a leukemia/lymphoma patient group that will not benefit from a TIRC7 modulatory treatment, according to method of any of claims 1 to 12.

18. The modulator of TIRC7 for use according to any of claims 13 to 17, wherein the patient is a relapsed or refractory leukemia/lymphoma patient in which a previous therapy with a compound selected from the group consisting of anti-CD20 antibody such as Rituximab, fludarabine and chlorodeoxiadenosine, has failed.

19. The modulator of TIRC7 for use according to any of claims 13 to 18, wherein the lymphoma is selected from Hodgkin lymphoma or non-Hodgkin lymphoma.

20. A method for treating a patient suffering from leukemia/lymphoma, the method comprising the steps of
(a) Determining the expression of TIRC7 in or on a tumor cell from the patient compared to a negative control,
(b) If the resultant of step (a) is that TIRC7 is expressed in or on the tumor cell,
(c) Administering to the patient a therapeutically effective dose of a TIRC7 modulator.

21. The method according to claim 20, wherein step (a) comprises a method according to any of claims 1 to 12.

22. The method according to claim 20 or 21, wherein the modulator of TIRC7 is an antibody binding to, and inhibiting, the extracellular domain of TIRC7, preferably wherein the antibody, or antibody derivatives of these molecules, such as chimerized, humanized, or otherwise optimized antibody molecules.

23. The method according to any of claims 20 to 22, wherein the lymphoma is selected from Hodgkin lymphoma or non-Hodgkin lymphoma.

24. The method according to any of claims 20 to 23, wherein the patient is a relapsed or refractory leukemia/lymphoma patient in which a previous therapy with a compound selected from the group consisting of anti-CD20 antibody such as Rituximab, fludarabine and chlorodeoxiadenosine, has failed.

25. The method according to any of claims 20 to 24, wherein the therapeutically effective dose of a TIRC7 modulator induces an increased caspase 3 dependent apoptosis in tumor cells in said patient.
